(19) United States
(12) Patent Application Publication
(10) Pub. No.: US 2022/0361738 A1
(43) Pub. Date: Nov. 17, 2022

(54) Method of soft tissue imaging system by different combinations of light engine, camera, and modular software

(71) Applicant: Cheng Chen, Greenville, NC (US)

(72) Inventor: Cheng Chen, Greenville, NC (US)

(21) Appl. No.: 17/807,727

(22) Filed: Jun. 19, 2022

(51) Int. Cl. A61B 1/06; A61B 1/05; A61B 5/1455; H04N 5/33

(52) U.S. Cl. CPC A61B 1/0638; A61B 1/05; A61B 5/14551; H04N 5/332

(57) ABSTRACT

Architecture and methodology of imaging systems are provided for multispectral tissue imaging with various embodiments. The architectural designs comprise hardware of multispectral light engines and cameras and software of image acquisition, processing, modeling, visualization, and quantification. Embodiments of imaging hardware in a medical device can include a light engine of multiple sources for noncoherent light for visible and fluorescence imaging and coherent light of very narrow bandwidths for laser speckle imaging. The imaging software can include anatomical imaging by visible light, blood perfusion imaging by fluorophores in blood, blood flow distribution imaging by light of high coherence, blood oxygen saturation imaging by light absorption in tissues and tissue composition imaging by light scattering in tissues based on the radiative transfer model of light-tissue interaction. Form factors in medical devices include endoscopic, laparoscopic, arthroscopic devices in medical tower or robot systems, cart device, and handheld scanning or tablet devices.

### FIELD

The present inventive concept relates to projecting light of multiple wavelengths or multiple wavelength bands onto a target such as tissues or organs with embedded blood vessels and capturing multiple images simultaneously or sequentially for image processing, modeling, visualization, and quantification by various parameters as biomarkers.

### BACKGROUND

Soft tissue imaging by optical means has been gaining more and more interests in the medical field for its safety and cost-effectiveness. It includes visible and near-infrared (NIR) light imaging, narrow band imaging; fluorescence imaging; laser speckle imaging, laser doppler imaging; other soft tissue imaging such as oxygen saturation and composition imaging.

Multispectral technologies allow combining light of visible light and NIR wavelengths during the imaging process and provide benefits of visualizing anatomical structure and quantitively visualizing distribution of functional/physiologic/compositional characteristics of organs and tissues.

### SUMMARY

Some embodiments of the present inventive concept provide several light engine designs for multispectral illumination. The method includes modular design of each light source in a light engine which can be of free space optics, or fiber optics coupling light emitting devices such as lasers, LEDs, noncoherent lamps etc. Each light source can be coherent, or non-coherent depending on the imaging application and processing requirement. Other optics characters of each light source such as power, irradiance and flux can be adjusted depending on the imaging application.

Some embodiments of the present inventive concept provide several camera designs for multispectral sensing. The method includes modular design for detecting separately light from a target in different wavelengths or wavelength bands which can be simultaneous and/or sequentially over these wavelengths or wavelength bands. The designs can include multi-sensor or single sensor with multispectral pixels or pixel regions or single sensor to detect each selected wavelength or wavelength band at a chosen time. The spectral regions of illumination and detection can range, for example, from 350 nm to 1050 nm which is determined by the spectral sensitivity of chosen sensor.

Some embodiments of the present inventive concept require innovative software architectural optimization based on selected multispectral illumination and camera sensing designs. Software flowchart includes image acquisition, processing, modeling, visualization, and quantification.

Some embodiments of the present inventive concept provide optimization of a list of imaging modules based on selected multispectral illumination and camera sensing designs. Imaging modules in a medical device include visible and NIR light imaging, narrow bandwidth light imaging; fluorescence imaging; laser speckle imaging, laser doppler imaging; other soft tissue imaging such as oxygen saturation and tissue composition imaging.

Some embodiments of the present inventive concept provide optimization of device form factors based on multispectral illumination and camera sensing designs. Form factors of medical devices include endoscopic/laparoscopic/arthroscopic devices for medical towers or robots, cart device with extension arm and camera head, and handheld scanning or tablet device.

### BRIEF DESCRIPTION OF THE DRAWINGS

FIG. 1 is a block diagram of the multispectral imaging system architecture in accordance with some embodiments of the present inventive concept(s).

FIG. 2 is a block diagram of the multispectral light engine design # 1 in accordance with some embodiments of the present inventive concept(s).

FIG. 3 is a block diagram of the multispectral light engine design # 2 in accordance with some embodiments of the present inventive concept(s).

FIG. 4 is a block diagram of the multispectral light engine design # 3 in accordance with some embodiments of the present inventive concept(s).

FIG. 5 is a block diagram of the multispectral light engine design # 4 in accordance with some embodiments of the present inventive concept(s).

FIG. 6 is a block diagram of the multispectral camera design # 1 in accordance with some embodiments of the present inventive concept(s).

FIG. 7 is a block diagram of the multispectral camera design # 2 in accordance with some embodiments of the present inventive concept(s).

FIG. 8 is a block diagram of the multispectral camera design # 3 in accordance with some embodiments of the present inventive concept(s).

FIG. 9 is a block diagram of the multispectral imaging software architecture in accordance with some embodiments of the present inventive concept(s).

FIG. 10 is a block diagram of the multispectral imaging form factor in accordance with some embodiments of the present inventive concept(s).

### DETAILED DESCRIPTION OF EMBODIMENTS

Embodiments of the present inventive concept will now be described more fully hereinafter with reference to the accompanying figures, in which some embodiments of the inventive concept are shown. This inventive concept may, however, be embodied in many different forms and should not be construed as limited to the embodiments set forth herein. Like numbers refer to like elements throughout. In the figures, layers, regions, elements or components may be exaggerated for clarity. Broken lines illustrate optional features or operations unless specified otherwise.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the inventive concept. The sequence of operations (or steps) is not limited to the order presented in the claims or figures unless specifically indicated otherwise.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

As will be appreciated by one of skill in the art, embodiments of the present inventive concept may be embodied as a method, system, data processing system, or computer program product. Accordingly, the present inventive concept may take the form of an embodiment combining software and hardware aspects, all generally referred to herein as a “circuit” or “module.” Furthermore, the present inventive concept may take the form of a computer program product on a non-transitory computer usable storage medium having computer usable program code embodied in the medium. Any suitable computer readable medium may be utilized including hard disks, CD ROMs, optical storage devices, or other electronic storage devices.

Computer program code for carrying out operations of the present inventive concept may be written in an object oriented programming language such as Matlab, Mathematica, Java, Smalltalk, C or C++. However, the computer program code for carrying out operations of the present inventive concept may also be written in conventional procedural programming languages, such as the “C” programming language or in a visually oriented programming environment, such as Visual Basic.

Certain of the program code may execute entirely on one or more of a user's computer, partly on the user's computer, as a stand alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

The inventive concept is described in part below with reference to flowchart illustrations and/or block diagrams of methods, devices, systems, computer program products and data and/or system architecture structures according to embodiments of the inventive concept. It will be understood that each block of the illustrations, and/or combinations of blocks, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the block or blocks.

These computer program instructions may also be stored in a computer readable memory or storage that can direct a computer or other programmable data processing apparatus to function in a particular manner, such that the instructions stored in the computer-readable memory or storage produce an article of manufacture including instruction means which implement the function/act specified in the block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the block or blocks.

Animal and human organs are composed of different types of soft and hard tissues. The soft tissues have complex structures and compositions. As the largest organ of the human body, for example, skin possess a layered structure of multiple tissues that include epidermis, dermis and hypodermis. The skin dermis consists of connective tissues, blood, endothelium and subendothelial connective tissues of blood vessels, fat, etc. To accurately model soft tissue imaging by optical means, one often applies the radiative transfer (RT) theory to quantify the light-tissue interaction. With the RT theory, one may quantify the optical response of imaged tissues by the following RT equation

s·∇L(r, s)=−(μₐ+μₛ)L(r, s)+μₛ∫_{4π}p(s, s′)L(r, s′)dω′+S(r, s) Eqn. (1)

where s is an unit vector in the direction of light propagation, L(r, s) is the radiance (W·sr⁻¹·m⁻²) describing the power flux propagating at r position and along s direction per unit solid angle, S(r, s) represents a directional light source density (W·sr⁻¹·m⁻³) contributing to L(r, s). In fluorescence imaging, S may be used to model the fluorophores in blood that are excited by the incident light. In other cases, such as tissue imaging by light of narrow wavelength band, one may ignore the S term if the medium is source-free. The optical parameters defined in Eqn. (1) for a particular type of tissues such as blood consists of absorption coefficient μₐ, scattering coefficient μₛ and single-scattering phase function p(s, s′). For modeling light-tissue interaction in all embodiments of the inventive concept in this application, we assume the phase function p(s, s′) can be replaced by a single-parameter function first proposed by Henyey and Greenstein in their classic paper published in 1941

$${p}_{H G} ( cos θ ) = \frac{1 - {g}^{2}}{4 {π ( 1 + {g}^{2} - 2 g cos θ )}^{\frac{3}{2}}} \qquad Eqn . ( 2 )$$

where cosθ=s·ss′ and g is the mean value of cosθ. With the above assumption, the optical parameters for characterization of light-tissue interaction by the RT theory consist of μₐ, μₛ and g. We note that these parameters are function light wavelength and tissue types.

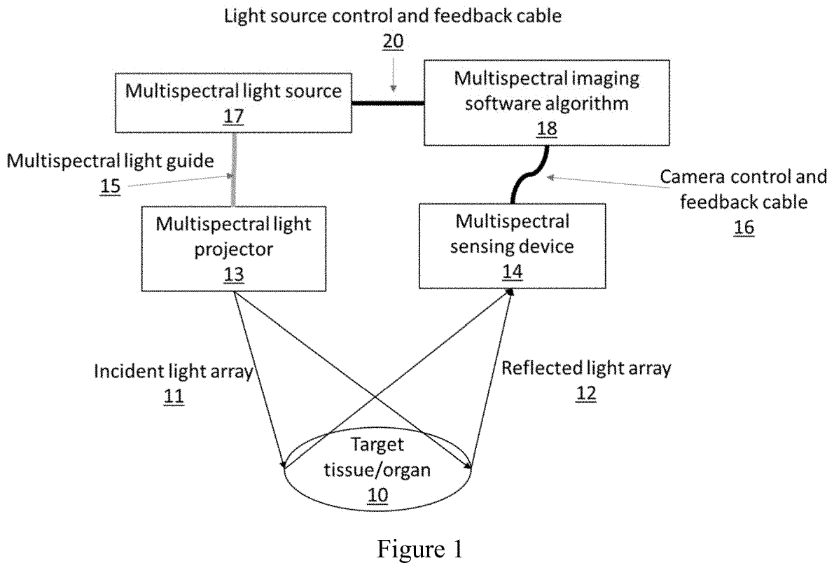

Referring first to FIG. 1, a system design architecture for soft tissue imaging in accordance with some embodiments of the present inventive concept will be discussed. Multispectral light source 17 generates wavelengths or wavelength bands 1 to N and passes the light through a light guide 15. The light guide 15 may include but not limited to fiber bundle, single-mode or multi-mode fiber, light pipe and/or other light transmitting components. The multispectral light projector 13 homogenizes and expands the beam of wavelengths 1 to N (11) and projects them onto a target such as tissues and organs 10. The multispectral light projector 13 may include but not limited to collimator, diffuser, homogenizer, combiner, fiber bundle and other light expanding components. The reflected or emitted light 12 from target such as tissues and organs is collected by multispectral sensing device 14 which may include but not limited to rigid or flexible endoscopic/laparoscopic/arthroscopic device, camera lens, adaptors, dichroic mirrors, prims, filters, and other beam splitting and combining components, CCD and CMOS sensor(s) and electronic device for control and data acquisition. The multispectral image processing software 18 controls multispectral light source 17 and multispectral sensing device 14 through cables or wireless means such as bluetooth (16, 20). The multispectral image processing software 18 performs functions such as image acquisition, processing, modeling, visualization, and quantification.

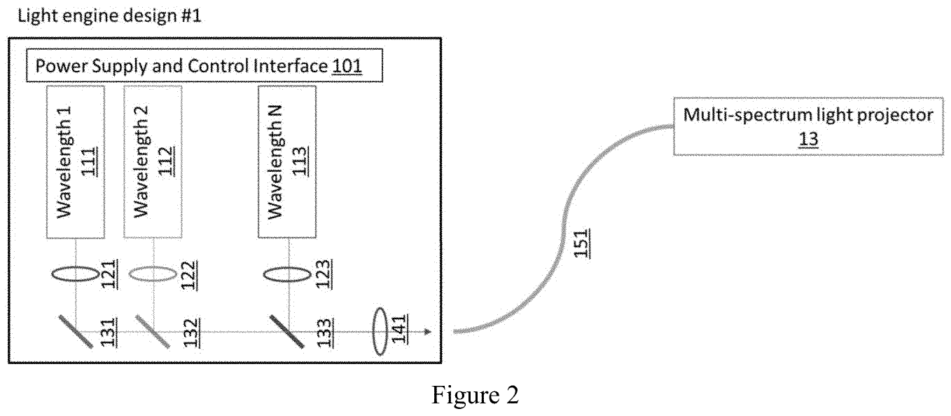

Referring to FIG. 2, a light engine design in accordance with some embodiments of the present inventive concept will be discussed. Wavelength or band 1 (111), Wavelength or band 2 (112), to wavelength or band N (113) are generated in forms of beams in free space, focused by optics lens and/or mirrors and/or other optics components (121, 122, 123) and aligned by dichroic mirrors, hot mirrors and/or other optics components (131, 132, 133). Then the beam is refocused by optics lens and/or mirrors and/or other optics components (141) and enters fiber bundle 151 and multispectral light projector 13. The light intensity, pulsing and other characteristics are controlled by power supply and software control interface 101. The total light power for each wavelength is calculated using the following equation:

P_{Total}=α×[α₁×P₁(λ₁, T₁) + . . . +αᵢ×Pᵢ(λᵢ, Tᵢ)+ . . . +α_{N}×P_{N}(λ_{N}, T_{N})] Eqn. (3)

where P_{Total} is the total light intensity emitted from multispectral light projector 13; Pᵢ(λᵢ, Tᵢ) is the power emitted from the source of λ^{th} wavelength or band λᵢ , for example, 112; Tᵢ is a pulsing parameter controlling how long the illumination of wavelength λᵢ is turned on; αᵢ is an attenuation parameter due to loss of the light intensity from optics components such as 122, 132 which are different for each light source of wavelength λᵢ; α is an attenuation parameter due to loss of the light intensity from optics components such as 141, 151, 13 which are the same for all wavelengths.

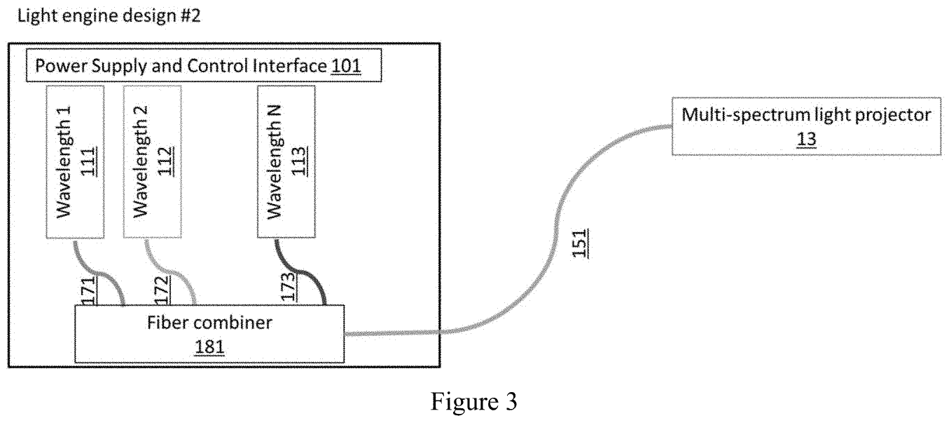

Referring to FIG. 3, another light engine design in accordance with some embodiments of the present inventive concept will be discussed. Wavelength or band 1 (111), Wavelength or band 2 (112), to wavelength or band N (113) are generated through fiber coupled form, transmitted by fibers and/or other optics components (171, 172, 173) and combined by fiber combiner and/or other optics components 181. Then the beam enters fiber bundle 151 and multispectral light projector 13. The light intensity, pulsing and other characteristics are controlled by power supply and control software interface 101. The fiber combiner 181 may include but not limited to split fibers, fused fibers, filters, and other optics coupling devices. Eqn. 3 applies to this design also.

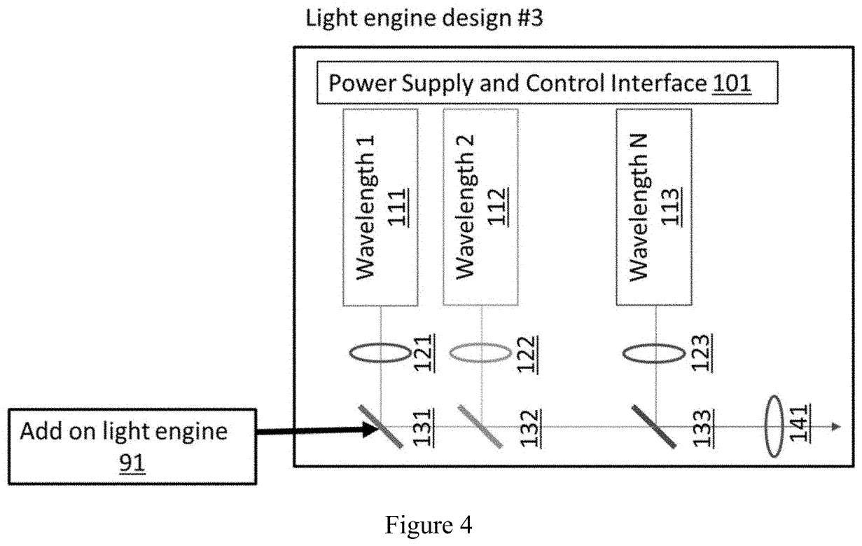

Referring to FIG. 4, another light engine design in accordance with some embodiments of the present inventive concept will be discussed. Additional wavelength can be added into light engine design # 1 (FIG. 2) through one or multiple modular addon light engines 91.

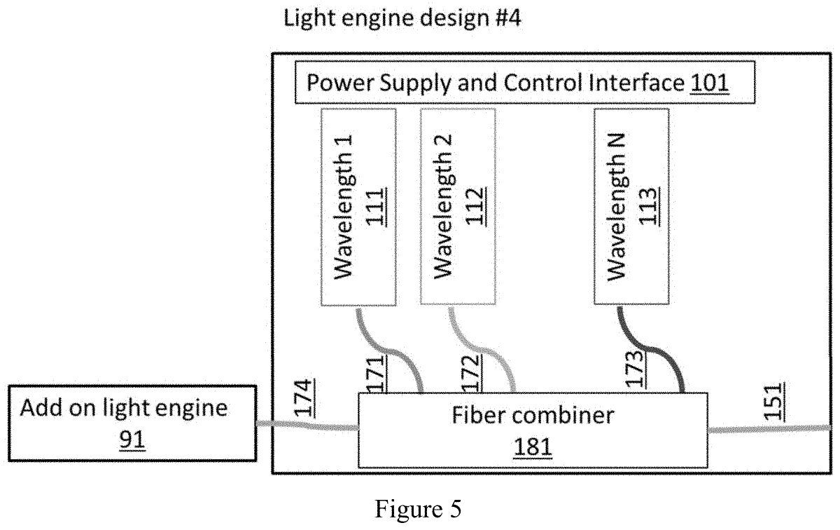

Referring to FIG. 5, another light engine design in accordance with some embodiments of the present inventive concept will be discussed. Additional wavelength can be added into light engine design # 2 (FIG. 3) through one or multiple modular addon light engines 91.

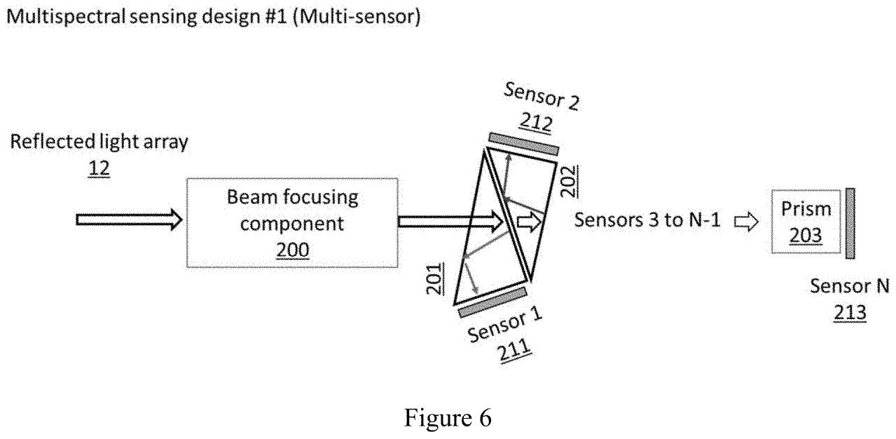

Referring now to FIG. 6, a multispectral sensing design in accordance with some embodiments of the present inventive concept will be discussed. A multi-sensor camera is used to detect reflected light with wavelength 1 (111) through prism and/or dichroic mirror 201 and sensor 1 (211), wavelength 2 (112) through prism and/or dichroic mirror 202 and sensor 2 (212), wavelength N (113) through prism and/or dichroic mirror 203 and sensor N (213). A beam focusing component 200 is used to collect the reflected light array 12 before it enters the camera system. The beam focusing component 200 may include but not limited to rigid or flexible endoscopic/laparoscopic/arthroscopic device, camera lens, adaptors and other optics beam collecting components. The image captured by the i^{th} sensor is defined using the following equation:

Img_{sensor i}=Img(λᵢ, Pᵢ, tᵢ, gᵢ, x, y) Eqn. (4)

Where is the λ^{th} wavelength for example 112; Pᵢ is the light power emitted by the i^{th} wavelength source; t is the i^{th} sensor exposure time; g is the i^{th} sensor gain; x is the horizonal pixel coordinate, y is the vertical pixel coordinate.

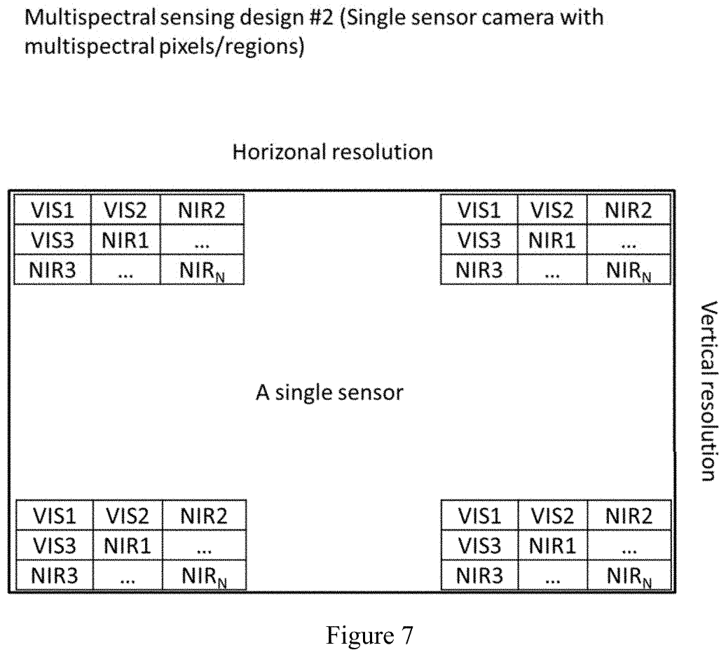

Referring to FIG. 7, another multispectral sensing design in accordance with some embodiments of the present inventive concept will be discussed. A single sensor camera with multispectral pixels/regions is described as
VIS1 represents a group of pixels that detect visible wavelength 1 for example red color light;
VIS2 represents a group of pixels that detect visible wavelength 2 for example green color light; VIS3 represents a group of pixels that detect visible wavelength 3 for example blue color light; NIR1 represents a group of pixels that detect near infrared wavelength 1 for example 700 nm-800 nm; NIR2 represents a group of pixels that detect near infrared wavelength 2 for example 800 nm-900 nm; NIR3 represents a group of pixels that detect near infrared wavelength 3 for example 900 nm-1000 nm; NIRN represents a group of pixels that detect near infrared wavelength N for example above 1000 nm.
The image captured for the i^{th} wavelength is defined using the following equation:

Imgᵢ=Img(λᵢ, Pᵢ, t, g, x/N, y/M) Eqn. (5)

Where is the i^{th} wavelength for example 112; Pᵢ is the light power emitted by the i^{th} wavelength source; t is the sensor exposure time; g is the sensor gain; x is the horizonal pixel coordinate, y is the vertical pixel coordinate; N is the horizonal image resolution resampling factor based on the layout of visible and near infrared pixels; M is the vertical image resolution resampling factor based on the layout of visible and near infrared pixels. The image for each wavelength has lower resolution than the original resolution of the sensor. The total number of effective pixels of each wavelength is defined by the following equation:

$${X}_{i} = \frac{X}{N} ; {Y}_{i} = \frac{Y}{M} \qquad Eqn . ( 6 )$$

Where X is the horizonal resolution of the original sensor; Y is the vertical resolution of the original sensor; Xᵢ is the horizonal resolution of the image for i^{th} wavelength; Yᵢ is the vertical resolution of the image for i^{th} wavelength. FIG. 7 is only a specific example, the number and layout of visible pixels (VIS1 to VIS3) and near infrared pixels (NIR₁ to NIR_{N}) can be different from FIG. 7 when a specific sensor is used.

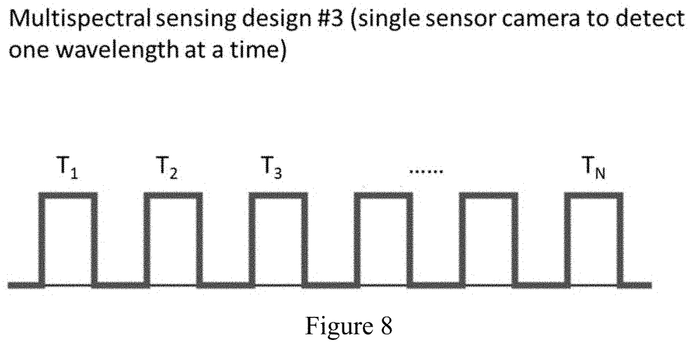

Referring to FIG. 8, another multispectral sensing design in accordance with some embodiments of the present inventive concept will be discussed. A single sensor camera to detect one wavelength at a time uses a series of pulse train to trigger one of N wavelengths (or multiple wavelengths at a time) and synchronize the single sensor with the light source for detection. A single sensor camera to detect one wavelength at a time is described as

T₁: wavelength 1 is detected, and wavelengths 2-N are not detected

T₂: wavelength 2 is detected, and wavelengths 1, 3-N are not detected

T₃: wavelength 3 is detected, and wavelength 1-2, 4-N are not detected

T_{N}: wavelength N is detected, and wavelength 1 to N-1 are not detected
The image captured by the sensor is defined using the following equation:

Img=Img₁(λ₁, P₁, T₁, t₁, g₁, x, y) + . . . + Imgᵢ(λᵢ, Pᵢ, Tᵢ, tᵢ, gᵢ, x, y)+ . . . + Img_{N}(λ_{N}, P_{N}, T_{N}, t_{N}, g_{N}, x, y) Eqn (7)

Where λᵢ is the i^{th} wavelength for example 112; Pᵢ is the light power emitted by the i^{th} wavelength source; t is the i^{th} sensor exposure time; g is the it^{h} sensor gain; x is the horizonal pixel coordinate, y is the vertical pixel coordinate; Tᵢ is a pulsing parameter controlling how long the wavelength is turned on. When a single sensor is used to detect a wavelength at a chosen time or multiple wavelengths at a chose time, additional optics such as notch filter, band pass filter may be needed for a specific imaging module such as fluorescence imaging.

Combination of multispectral sensing design can be made through multi-sensor (FIG. 6) can be triggered by a pulse train signal (FIG. 8); single sensor with multispectral pixels/regions (FIG. 7) can be triggered by a pulse train signal (FIG. 8).

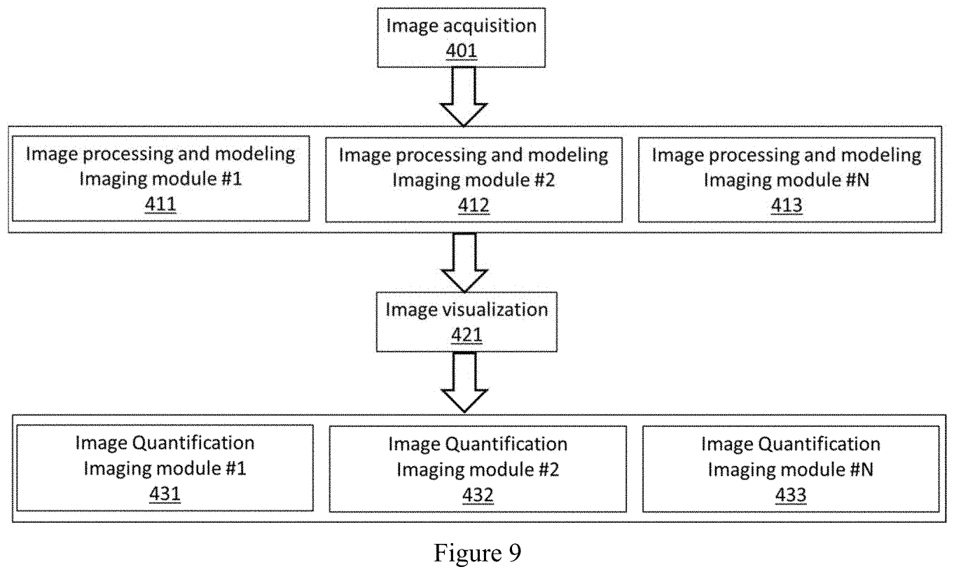

Referring first to FIG. 9, software architecture for multispectral imaging in accordance with some embodiments of the present inventive concept will be discussed. Image acquisition unit 401 acquires raw multispectral image sequence and adjust brightens, contrast, color balance and gamma value. Image processing and modeling units (411, 412, 413) may include but not limited to calculating the following results from raw sequence using artificial intelligence driven algorithms:
Visible light imaging, narrow band imaging; Fluorescence imaging
Laser speckle imaging, laser doppler imaging; Other soft tissue imaging such as oxygenation imaging, oxygen saturation imaging
Image visualization unit 421 may include but not limited to the following functions:
   Use a specific color map to create a pseudo mapping for the result images; Display multiple images at different locations on a screen; Display multiple images in an overlay setting with adjustable transparency; Other features such as glare reduction
Image quantification unit (431, 432, 433) may include but not limited to the following functions using artificial intelligence driven algorithms and machine learning algorithms:
   Intra image comparison/quantification: Compare one ROI (region of interest) with another ROI of the same image and quantify the comparison result; Inter images comparison/quantification: Compare one image (or ROI of one image) with anther image (or ROI of another image) of the same case or different cases but the same patient and quantify the comparison result.

The modular light engine, sensing and software designs addressed above allow a variety of form factors while applying multispectral soft tissue imaging to a medical device. For example, multispectral light projector (13 in FIG. 1) and sensing device (14 in FIG. 1) can be combined into an endoscopic/laparoscopic/arthroscopic chip-on-tip technology or a scope assembly with camera adaptor and camera.

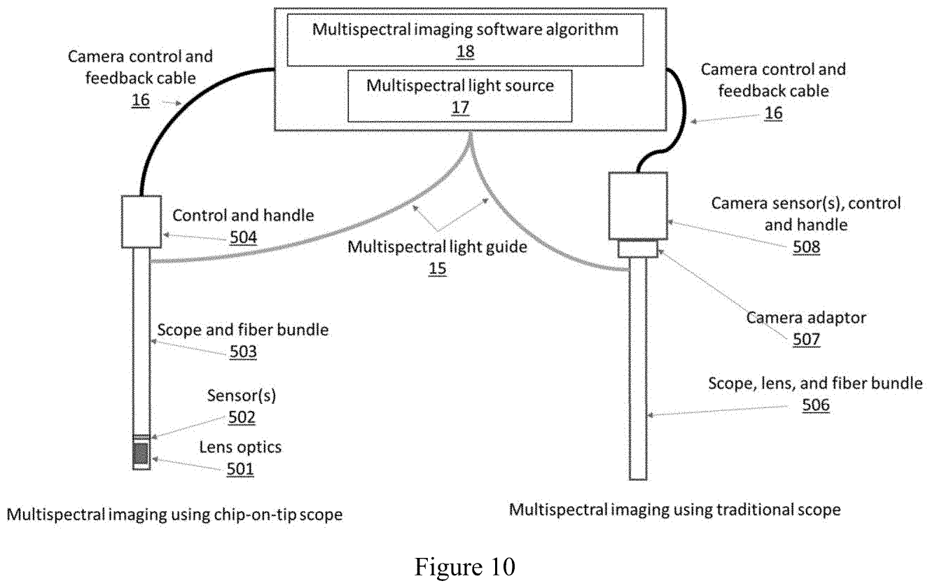

Referring first to FIG. 10, one of the form factors for multispectral imaging in accordance with some embodiments of the present inventive concept will be discussed. On the left is a diagram for multispectral chip-on-tip scope. Multispectral light is emitted through multispectral light source 17, light guide 15, scope and fiber bundle 503. The reflected light is captured through lens optics 501, sensor(s) on the tip of the scope 502. The images are processed by multispectral imaging software algorithm 18 and controlled by control and handle 504. The chip-on-tip sensor(s) 502 may use one of the multispectral sensing designs addressed above in FIG. 6, FIG. 7, FIG. 8 or a combination of them to achieve multispectral software tissue imaging. On the right is a diagram for traditional scope design with camera and adaptor. Multispectral light is emitted through multispectral light source 17, light guide 15, scope, lens, and fiber bundle 506. The reflected light is captured through scope, lens, and fiber bundle 506, camera adaptor 507 and camera sensor(s) 508. The images are processed by multispectral imaging software algorithm 18. The camera sensor(s) 508 may use one of the multispectral sensing designs addressed above in FIG. 6, FIG. 7, FIG. 8 or a combination of them to achieve multispectral software tissue imaging.

The form actors of multispectral imaging device may include but not limited to
Endoscopic/Laparoscopic/Arthroscopic device (medical tower or robot); Cart device with extension arm and camera head; Handheld scanning or tablet device

That which is claimed is:

1. A multispectral imaging system, the system comprising:
A multispectral light engine that emits and combines light of N different wavelengths or wavelength bands through free space optics and/or fiber coupling optics;
A multispectral sensing device images light from imaged tissues of N different wavelengths or wavelength bands through multi-sensor and/or single sensor optics; and
A multispectral imaging software acquires, processes, models, visualizes and quantifies images of N different wavelengths or wavelength bands through optical light-tissue modeling algorithms according to Eqns (1) and (2), artificial intelligence algorithms, machine learning algorithms and image fusion algorithms.

2. A multispectral light engine emits and combines light of N different wavelengths or wavelength bands through free space optics and/or fiber coupling optics that is defined in Eqn. (3).

3. A multispectral light engine emits and combines light of N different wavelengths or wavelength bands from multiple addon light sources.

4. A multispectral sensing device images light of N different wavelengths or wavelength bands through multi-sensor design defined in Eqn. (4) and/or single sensor camera with multispectral pixels/regions design defined in Eqn. (5) and Eqn. (6) and/or single sensor camera to detect one wavelength at a time defined in Eqn. (7) and/or a combination of them.

5. The method of claim 2, wherein the multispectral illumination is embodied using chip-on-tip endoscopic/laparoscopic/arthroscopic technology and wherein the multispectral illumination is embodied using endoscopic/laparoscopic/arthroscopic scope, camera adaptor and camera assembly.

6. The method of claim 3, wherein the multispectral add-on modular illuminations are embodied using chip-on-tip endoscopic/laparoscopic/arthroscopic technology and wherein the multispectral add-on modular illuminations are embodied using endoscopic/laparoscopic/arthroscopic scope, camera adaptor and camera assembly.

7. The method of claim 4, wherein the multispectral sensing is embodied using chip-on-tip endoscopic/laparoscopic/arthroscopic technology and wherein the multispectral sensing is embodied using endoscopic/laparoscopic/arthroscopic scope, camera adaptor and camera assembly.

8. A multispectral imaging software acquires, processes, models, visualizes and quantifies images of N different wavelengths or wavelength bands through optical light-tissue modeling algorithms according to Eqns (1) and (2), artificial intelligence algorithms, machine learning algorithms and image fusion algorithms.

9. The method of claim 8, wherein the multispectral image software is embodied using chip-on-tip endoscopic/laparoscopic/arthroscopic technology and wherein the multispectral image software is embodied using endoscopic/laparoscopic/arthroscopic scope, camera adaptor and camera assembly.

10. The method of claim 8, wherein the multispectral image processing is embodied using Monte Carlo simulations to numerically model light-tissue interaction according to Eqns (1) and (2) and identify tissue compositions by their respective optical parameters of μₐ, μₛ and g as functions of wavelength and wherein the multispectral image processing is embodied using Monte Carlo simulations to numerically model light-tissue interaction in tissues and blood according to Eqns (1) and (2) and identify the ratio of oxygenated and deoxygenated red blood cells in blood by their respective absorption coefficient μₐ as functions of wavelength for determination of oxygen saturation of the blood.
